(19) United States
(12) Patent Application Publication
(10) Pub. No.: US 2004/0191208 A1
(43) Pub. Date: Sep. 30, 2004

(54) Cosmetic composition for men's skin care and hair care

(75) Inventor: Olivier Courtin, Boulogne-Sur-Seine (FR)

Correspondence Address: IP DEPARTMENT OF PIPER RUDNICK LLP, ONE LIBERTY PLACE, SUITE 4900, 1650 MARKET ST, PHILADELPHIA, PA, 19103, US

(73) Assignee: Laboratoires Clarins, a corporation of France, Neuilly-sur-Seine (FR)

(21) Appl. No.: 10/806,536

(22) Filed: Mar. 23, 2004

Related U.S. Application Data
(63) Continuation of application No. PCT/FR02/03256, filed on Sep. 24, 2002.

(30) Foreign Application Priority Data
Sep. 24, 2001 (FR) FR 01/12270

(51) Int. Cl. A61K 35/78; A61K 7/06

(52) U.S. Cl. 424/74; 424/750; 424/756; 424/764

(57) ABSTRACT

A cosmetic composition including a water-soluble extract of galangal (Alpinia officinarum), a water-soluble extract of buffalo grass (Hierochloe odorata), and a water-soluble extract of purslane (Portulaca oleracea), and a men's skin care product, a shaving cream product, a sunless tanning product, a men's hair care product and a men's after shave product.

### RELATED APPLICATION

This is a continuation of International Application No. PCT/FR02/03256, with an international filing date of Sep. 24, 2002 (WO 03/026605, published Apr. 3, 2003), which is based on French Patent Application No. 01/12270, filed Sep. 24, 2001.

### FIELD OF THE INVENTION

This invention pertains to the field of cosmetology. It pertains to a cosmetic composition most particularly adapted for men's skin and hair care.

### BACKGROUND

Men's skin has specific characteristics that are different from women's skin. In men, the cells of the epidermis are more numerous and active and, at the level of the dermis, the fibroblasts are more active and collagen is more abundant. This is manifested by a thicker epidermis, a denser dermis, and intense cellular and biological activities.

Male hormones stimulate sebaceous secretion and hair follicles. More abundant sebaceous secretion makes men's skin shinier with dilated pores. One consequence of this abundant sebaceous secretion is often severe acne at puberty. A cosmetic composition for the care of men's skin should, therefore, provide a pure, unshiny appearance. The texture of the cosmetic compositions for men should also be fluid, nonoily and not promote the appearance of comedos.

Men's skin is richer in collagen and their dermis is thicker. The sebaceous secretion protects their skin against attack. For these reasons, men's skin retains a youthful appearance for a longer period of time, but ages abruptly, with the less numerous wrinkles being more pronounced and deeper.

Moreover, hormonal stimulation of hair follicles necessitates shaving on a daily basis for 96% of men. This aggressive activity irritates and dries men's skin by destroying the hydrolipid film. A cosmetic composition for the care of a man's skin more particularly adapted to shaving should not only reduce razor bum, but also soothe, hydrate, prevent and limit the risk of infection linked to razor “nicks.”

Cosmetic compositions suitable for use in the care of men's skin should provide freshness and have a draining action to attenuate red blotches on the skin and formation of pouches under the eyes.

### SUMMARY OF THE INVENTION

This invention relates to a cosmetic composition including a water-soluble extract of galangal (Alpinia officinarum), a water-soluble extract of buffalo grass (Hierochloe odorata), and a water-soluble extract of purslane (Portulaca oleracea).

This invention also relates to a men's skin care product, a shaving cream product, a sunless tanning product, a men's hair care product and a men's after shave product.

### DETAILED DESCRIPTION

This invention provides a new cosmetic composition responding specifically to the needs of men's skin as described above. The cosmetic composition is remarkable in that it comprises energizing, soothing and sanitizing plant-derived active principles.

The cosmetic compositions of the invention comprise:

a water-soluble extract of galangal (Alpinia officinarum),

a water-soluble extract of buffalo grass (Hierochloe odorata), and

a water-soluble extract of purslane (Portulaca oleracea).

The water-soluble extracts contained in the cosmetic compositions are preferably hydroglycolic extracts.

Galangal (Alpinia officinarum) of the Zingiberaceae family is a shrub originating in East Asia whose black, gnarled rhizome resembles that of ginger. Galangal is also called Chinese ginger. It is widely used as a condiment especially in Indonesia where it is indispensable for traditional cuisine. Galangal is also part of the Chinese pharmacopoeia not only for its digestive virtues, but especially for its energizing qualities. In application on the skin, it has long been known to bestow energy and vitality. The galangal rhizome is rich in active principles with stimulating and reinvigorating properties such as essential oils, flavonoids, minerals, sugars, etc.

Thus, the water-soluble galangal extract used in the compositions of the invention is an extract containing flavonoids. This extract is advantageously prepared from galangal rhizomes. The galangal extract is obtained by controlled extraction with a water/propylene glycol mixture followed by a centrifugation/filtration step. The galangal extract is rich in essential oils such as bomeol, cineol, caryophyllene and geraniol. It also contains phytosterols and flavonoids such as galangin and kaempferol. The flavonoid content is on the order of 1.5 g/l (rutin equivalent). The flavonoids and principally the galangin contained in the galangal extract have an antifungal and antibacterial activity useful for combating cutaneous disorders.

The cosmetic compositions of the invention also contain a water-soluble extract of buffalo grass (Hierochloe odorata). Buffalo grass (Hierochloe odorata) of the Poaceae family is native to Europe and North America. This grass of the cold and humid zones (above latitude 40°) terminates its growth at the end of summer and can reach up to 60 cm in height. Equipped with an extensive, rampant and rather deep root system, the plant reproduces itself essentially by propagation of the rhizome, since the seeds are small in number and often infertile. From one year to the next, the new sprouts appear from the faded foliage of the previous year, first giving rise to fructiferous stalks surrounded by short foliage quickly giving way to a new sterile basal sprout of rapid development thus yielding the plant that is usually harvested.

Buffalo grass has a particularly agreeable odor, similar to that of vanilla, which develops after flowering. Consequently, because of its delicate odor, it is widely used at present in candies, drinks and perfumes. Furthermore, buffalo grass is recommended in infusion form for relieving coughs, sore throats and chapping. It can also be used as an ocular rinse. Its high richness in coumarins and umbelliferons, powerful stimulators of cellular respiration, make buffalo grass extract particularly suitable for the specific requirements of men's skin by bestowing on it venotonic and anti-inflammatory qualities.

The water-soluble extract of buffalo grass used in the invention is an extract containing coumarins and umbelliferons. It is obtained by grinding the aerial parts of the plant prior to maceration in ethanol alcohol. The extract is filtered, then purified and, finally, undergoes evaporation prior to being incorporated in an excipient of the glycerin type.

The cosmetic compositions of the invention contain a water-soluble extract of purslane (Portulaca oleracea) in addition to the water-soluble extract of galangal (Alpinia officinarum). Purslane (Portulaca oleracea) is a plant originally from India that was introduced into Europe by the English in the 16^{th} century. This small, fatty plant has rubbery stalks that become engorged with water like the thick, fleshy leaves. Purslane is one of the oldest representatives of our popular flora which is used not only in salads and as a condiment, but also as a medicinal plant. Louis XIV's gardener considered purslane to be a health-food salad and it still today comprises the base of the “Crétois” diet because of its high content of omega-3 fatty acids. In the therapeutic field, purslane has been used since ancient times as a medication for the gastrointestinal and respiratory tracts. In topical application, it is known for its anti-inflammatory and soothing properties. It is indeed these latter properties that justify its use in cosmetology, most particularly for skin care for men since it can combat razor burn, calm irritations and soothe red blotches.

The water-soluble extract of purslane used in the invention is an extract containing omega-3 fatty acids. This extract is advantageously obtained from the entire plant. Extraction is performed after washing the plants, followed by a filtration step. Two purification steps are then performed prior to a concentration step.

The cosmetic compositions according to the invention advantageously comprise:

from about 0.1 to about 10% by weight, preferably about 0.5 to about 5% by weight, of water-soluble galangal extract;

from about 0.1 to about 10%, preferably about 0.5 to about 5%, of water-soluble buffalo grass extract; and

from about 0.1 to about 10%, preferably about 0.5 to about 5%, water-soluble purslane extract.

In addition to the water-soluble extracts of galangal, buffalo grass and purslane, the compositions of the invention can also contain other active plant extracts. The cosmetic compositions advantageously contain one or more plant extracts selected from: a gaultheria extract, a grindelia extract, an aloe extract, a sunflower extract and an extract of Mourera fluviatilis.

Gaultheria is a shrub with evergreen leaves that grows in the woods and glades of the eastern United States and Canada. It has analgesic, astringent, anti-inflammatory and stimulant properties. It contains about 0.15 to about 0.25% salicylic acid. This beta hydroxy acid is known for its keratolytic action that can reduce the thickness of the comeous layer and thus reduce the thicker skin appearance characteristic of men's skin.

The hydroglycolic extracts of gaultheria are obtained by percolation of the raw material in a suitable solvent system. Then, by filtration, concentration and atomization, extracts of the desired quality are obtained in liquid or dry form. Gaultheria extracts can be used in any cosmetic composition designed to soothe men's skin, such as creams to be used after sunbathing and aftershave lotions because of its astringent and anti-inflammatory properties.

Grindelia (Grindelia robusta) is a robust plant originally from California and the American Southeast. It resembles a large daisy and forms a thick clump from 50 to 90 cm in height. Its activity is based on a resin recovered from the entire plant. In external use and for cosmetic applications, grindelia extract can be used for its anti-inflammatory and antibacterial properties. Its use is notably based on the presence of diterpenes, phenolic compounds, gallic tannins, flavonoids and saponisides including β-escin which has an anti-edema and anti-inflammatory action and diminishes capillary fragility.

The cosmetic compositions of the invention can also contain an aloe extract as an anti-irritant, anti-dehydrating and conditioning agent. It is known that aloe leaves, after peeling and removing the spines, yield a gel that is especially rich in sugars and contain traces of magnesium lactate which has already found applications in the field of cosmetics because of its texture qualities and properties.

The extract of sunflower (Helianthus sp.) that can be particularly useful in the cosmetic compositions of the invention is the extract of sunflower stalks (Helianthus annus) rich in auxin described in French patent application No. 2,789,901. This extract has a tonic and firming effect. It is obtained by maceration of sunflower stalks in a water/glycerin mixture, that are dried and put in suspension in a butylene glycol/water mixture (volume/volume).

The extract of Mourera fluviatilis, a tropical plant adapted to the spring waters of the Amazon basin, present in the cosmetic compositions according to the invention, acts specifically on the epidermal corneous layers. It simultaneously exerts a moisture regulator effect, a substantial moisture content regulator effect, a dehydration reducing effect and a hydration prolonging effect. It acts by stopping dehydration due to imperceptible perspiration and equilibrating the hydration of the skin as a function of the relative humidity of the ambient air.

For the preparation of the extract of Mourera fluviatilis, the entire plant without the roots is ground. An aqueous extract is then created. The extract of Mourera fluviatilis is obtained after a purification/filtration step.

The compositions according to the invention can also comprise one or more formulation agents that are known and conventionally used in cosmetic compositions such as the following nonlimitative examples: conditioners, colorants, film-forming active principles, surface-active agents, perfumes, emulsifiers, oils, glycols, dihydroxyacetone and erythrulose. Those skilled in the art of cosmetics know that formulation agents can be added to the compositions of the invention to obtain a product such as the following examples: a body and hair shampoo, a facial cleaner, a shaving preparation agent, a hydrating gel or balm, a soothing lotion for the face, neck and shoulders, an eye contour cream, a deodorant or a sunless tanning product. When the cosmetic composition is used in the form of a sunless tanning product, the composition can also contain dihydroxyacetone and/or erythrulose. Dihydroxyacetone has been known since the 1960s as a sunless tanning agent. Erythrulose is a natural sugar having the chemical name of 1,3,4-trihydroxy-2 butanone. Its use in sunless tanning cosmetic compositions is described in French patent application No. 2,772,268.

The compositions according to the invention can be presented in any form known in the cosmetology field without other pharmaceutical restrictions than the water-soluble nature of the extracts of galangal, buffalo grass and purslane. The compositions according to the invention are advantageously presented in the form of gels, creams, balms, milks, foaming products and the like to respond principally to the need for application on men's hair, skin and the face.

The invention, therefore, pertains to the cosmetic use of a composition according to the invention for men's skin care. More particularly, the invention pertains to the cosmetic use of a composition according to the invention for the preparation of the skin for shaving. The invention also pertains to the cosmetic use of a composition according to the invention for the preparation of a sunless tanning product. The invention further pertains to the cosmetic use of a composition according to the invention for men's hair care.

Other advantages and characteristics of the invention will become apparent from the examples below which pertain to the anti-inflammatory activity of purslane extracts and examples of formulations which are presented in an illustrative manner and should not be interpreted as limiting the scope of the invention.

I Anti-inflammatory Activity of Purslane Extract

I.1 Principle

In the initial steps of the induction of inflammation, the arachidonic acid stored in the cell membranes is liberated. Once the arachidonic acid has been liberated, it can follow different metabolic pathways. The pathways include: the cyclooxygenase enzyme pathway which leads to the prostaglandins, and the lipoxygenase enzyme pathway which leads to the leukotrienes.

The anti-inflammatory activity of purslane extract was, therefore, tested in vitro by evaluating the inhibition of cyclooxygenase. The ELISA-based test employed made it possible to monitor the synthesis of prostaglandins (PGE₂) in cultures of human keratinocytes. The cells were used at subconfluence with a cellular multiplication and an active metabolic activity. Two approaches were employed: approach 1: synthesis of the PGE₂ intrinsic to the cultured cells; and approach 2: synthesis of the PGE₂ induced chemically by PMA (phorbol myristate acetate), an inflammatory agent.

I.2 Protocol

Normal human keratinocytes were cultured at 37° C. for 48 hours in a defined medium in the presence of the following products:

approach 1: -purslane extract (0.1%, 0.5%, 1% and 3% vol/vol) - aspirin 100 μM (anti-inflammatory, reference compound) approach 2: - purslane extract (0.1%, 0.5%, 1% and 3% vol/vol) - indomethacin 1 μM (anti-inflammatory, reference compound) - stimulation with PMA 1 μg/ml (after 24 hours of incubation).

At the end of 48 hours, the different media were collected for quantitative determination of the PGE₂.

I.3 Results

The cytotoxicity of purslane extract was evaluated prior to any of the studies of the influence of the extract on the synthesis of PGE₂. No cytotoxicity was found at the selected concentrations (from 0.1 to 3%).

The results obtained for approach 1 are summarized in Table 1 below.

TABLE 1

| | | | Purslane extract | | | |
|---|---|---|---|---|---|---|
| | Control | Aspirin | 0.1% | 0.5% | 1% | 3% |
| Pg PGE₂/ | 4959.9 | 1968.2 | 2199.8 | 2289.6 | 2260.7 | 1903.3 |
| mg prot | | | | | | |
| % control | 100 | 39.7 | 44.4 | 46.2 | 45.6 | 38.4 |
| % inhibition | | 60.3 | 55.6 | 53.8 | 54.4 | 61.6 |
| of the | | | | | | |
| liberation of | | | | | | |
| PGE₂ | | | | | | |

The purslane extract inhibits liberation of the prostaglandins produced naturally by the cultured cells from 53.8 to 61.6%. It should be noted that the reference compound used (aspirin) inhibits liberation of these prostaglandins by 60.3%.

The results obtained for approach 2 are summarized in Table 2 below.

TABLE 2

| | | Indo- | Purslane extract | | | |
|---|---|---|---|---|---|---|
| | Control | methacin | 0.1% | 0.5% | 1% | 3% |
| Pg PGE₂/ | 8010.5 | 1683.9 | 6404.2 | 5828.4 | 4339.4 | 2680.6 |
| mg prot | | | | | | |
| % control | 100 | 21.0 | 80 | 72.8 | 54.2 | 33.5 |
| % inhibition | | 79.0 | 20 | 27.2 | 45.8 | 66.5 |
| of the | | | | | | |
| liberation of | | | | | | |
| PGE₂ | | | | | | |

The protective capacity against the liberation of prostaglandins induced by the pro-inflammatory agent PMA was studied using approach 2.

There exists a preventive protective effect of purslane starting at 0.1% of use. At 3%, the purslane extract inhibits more than 65% of the secretion of PGE₂. Thus, purslane extract is an excellent biological attenuator of inflammation. Note the powerful effect of the reference compound indomethacin which inhibits the inflammatory response (80%) when used at 1 μM.

II Formulation Examples of Cosmetic Compositions

II.1 Formulation in the Form of a Hair and Body Shampoo

| | |
|---|---|
| Film-forming quaternary polymer | 0.2 |
| Sodium lauryl ether sulfate | 30.0 |
| Betaine | 6.0 |
| Glutamic acid salt | 10.0 |
| Sulfosuccinic acid salt | 10.0 |
| EDTA Na₂ | 0.1 |
| MIPA lauramide | 1.0 |
| Antistatic film-forming agent | 2.0 |
| Preservatives | 0.5 |
| Panthenol | 0.5 |
| Silicone oil | 1.0 |
| Perfume | 3.0 |
| Galangal extract | 1.0 |
| Purslane extract | 1.0 |
| Buffalo grass extract | 1.0 |
| Colorant | 0.1 |
| Purified water | qsp 100 |

II.2 Formulation in the Form of a Foaming Facial Cleanser

| | |
|---|---|
| Carboxy vinyl resin | 0.10 |
| Sodium lauryl ether sulfate | 12.00 |
| Film-forming quaternary polymer | 0.10 |
| Sodium lauryl sarcosinate | 10.00 |
| Cocoamphoacetate | 5.00 |
| EDTA Na₂ | 0.05 |
| Preservatives | 0.50 |
| Emulsifiers | 3.00 |
| Glycol distearate | 3.00 |
| Dimethicone | 1.00 |
| MIPA lauramide | 2.00 |
| Betaine | 10.00 |
| Shea butter | 0.50 |
| Galangal extract | 1.00 |
| Purslane extract | 1.00 |
| Buffalo grass extract | 1.00 |
| Grindelia extract | 1.00 |
| Gaultheria extract | 0.50 |
| Panthenol | 0.50 |
| Menthol | 0.30 |
| Perfume | 1.00 |
| Purified water | qsp 100 |

II.3 Formulation in the Form of a Shaving Gel

| | |
|---|---|
| Fatty acids | 12.00 |
| Triethanolamine | 7.00 |
| Perfume | 1.50 |
| Sorbitol | 4.00 |
| Galangal extract | 1.00 |
| Purslane extract | 1.00 |
| Buffalo grass extract | 1.00 |
| Grindelia extract | 1.00 |
| Aloe extract | 1.00 |
| Celluloses | 0.50 |
| EDTA Na₂ | 0.50 |
| Preservatives | 0.20 |
| Colorants | 0.30 |
| Purified water | qsp 100 |

II.4 Formulation in the Form of a Hydrating Facial Balm

| | |
|---|---|
| Emulsifiers | 7.50 |
| Isoparaffin | 6.00 |
| Fatty acid ester | 8.00 |
| Perhydrosqualene | 2.00 |
| EDTA Na₂ | 0.50 |
| Glycerin | 5.00 |
| Preservatives | 0.50 |
| Neutralized carboxy vinyl resin | 0.50 |
| MICA | 0.20 |
| Polyacrylic resin | 1.00 |
| Talc | 0.50 |
| Colorants | 0.15 |
| Galangal extract | 2.00 |
| Purslane extract | 3.00 |
| Buffalo grass extract | 2.00 |
| Sunflower extract | 1.00 |
| Mourera extract | 1.00 |
| Vitamins A and E | 0.20 |
| Perfume | 0.50 |
| Purified water | qsp 100 |

II.5 Formulation in the Form of a Sunless Tanning Milk

| | |
|---|---|
| Fatty alcohol | 1.00 |
| Preservatives | 0.60 |
| Fatty acid ester | 4.00 |
| Glycerol stearate | 4.00 |
| Glycerin | 5.00 |
| EDTA Na₂ | 0.05 |
| Silicone oil | 5.00 |
| Polyacrylic resin | 2.00 |
| Vitamins A and E | 0.50 |
| Galangal extract | 2.00 |
| Purslane extract | 3.00 |
| Buffalo grass extract | 2.00 |
| Perfume | 0.50 |
| Dihydroxyacetone | 4.00 |
| Erythrulose | 1.00 |
| Purified water | qsp 100 |

1. A cosmetic composition comprising:
a water-soluble extract of galangal (Alpinia officinarum),
a water-soluble extract of buffalo grass (Hierochloe odorata), and
a water-soluble extract of purslane (Portulaca oleracea).

2. The cosmetic composition according to claim 1, wherein the water-soluble extract of galangal is an extract containing flavonoids.

3. The cosmetic composition according to claim 1, wherein the water-soluble extract of buffalo grass is an extract containing coumarins and umbelliferons.

4. The cosmetic composition according to claim 1, wherein the water-soluble extract of purslane is an extract containing omega-3 fatty acids.

5. The cosmetic composition according to claim 1, comprising:
from about 0.1 to about 10% by weight of water-soluble galangal extract;
from about 0.1 to about 10% of water-soluble buffalo grass extract; and
from about 0.1 to about 10% of water-soluble purslane extract.

6. The cosmetic composition according to claim 1, comprising:
from about 0.5 to about 5% by weight of water-soluble galangal extract;
from about 0.5 to about 5% of water-soluble buffalo grass extract; and
from about 0.5 to about 5% of water-soluble purslane extract.

7. The cosmetic composition according to claim 1, further comprising one or more plant extracts selected from the group consisting of a gaultheria extract, a grindelia extract, an aloe extract, a sunflower extract and an extract of Mourera fluviatilis.

8. The cosmetic composition according to claim 1, further comprising one or more additives selected from the group consisting of conditioners, colorants, film-forming active principles, surface-active agents, perfumes, emulsifiers, oils, glycols, dihydroxyacetone and erythrulose.

9. The cosmetic composition according to claim 1, in the form of gels, creams, balms, milks or foaming products.

10. A men's skin care product comprising the composition according to claim 1.

11. A shaving cream product comprising the composition according to claim 1.

12. A sunless tanning product comprising the composition according to claim 1.

13. A men's hair care product comprising the composition according to claim 1.

14. A men's after shave product comprising the composition according to claim 1.
